(19) United States
(12) Patent Application Publication
(10) Pub. No.: US 2024/0164706 A1
(43) Pub. Date: May 23, 2024

(54) IN-VIVO OBSERVATION SYSTEM, OBSERVATION SYSTEM, IN-VIVO OBSERVATION METHOD, AND IN-VIVO OBSERVATION DEVICE

(71) Applicant: SONY GROUP CORPORATION, TOKYO (JP)

(72) Inventors: HIROYASU BABA, TOKYO (JP); SHINJI KATSUKI, TOKYO (JP); FUMISADA MAEDA, TOKYO (JP); JUN ARAI, TOKYO (JP); KOHEI AMANO, TOKYO (JP); SHO INAYOSHI, TOKYO (JP)

(21) Appl. No.: 18/550,608

(22) PCT Filed: Feb. 9, 2022
(86) PCT No.: PCT/JP2022/005101

§ 371 Date: Sep. 14, 2023

(30) Foreign Application Priority Data
Mar. 25, 2021 (JP) 2021-052353

(51) Int. Cl. A61B 5/00; A61B 1/00; A61B 1/06; A61B 1/313; H04N 25/47

(52) U.S. Cl. CPC A61B 5/48; A61B 1/00149; A61B 1/06; A61B 1/3132; A61B 5/0051; H04N 25/47

(57) ABSTRACT

An in-vivo observation system including: an excitation device (150) that vibrates an object in a living body; an event vision sensor (200) that detects, as an event, a change due to the vibration in a luminance value of light emitted from the object; and an estimation unit (528) that estimates a characteristic of the object on a basis of sensing data from the event vision sensor is provided.

### FIELD

The present disclosure relates to an in-vivo observation system, an observation system, an in-vivo observation method, and an in-vivo observation device.

### BACKGROUND

In recent years, by progress in an endoscope, it is becoming possible to realize minimally invasive non-laparotomy with a small wound and faster recovery after surgery compared to laparotomy.

### CITATION LIST

### Patent Literature

Patent Literature 1: JP 2017-53890 A

### SUMMARY

### Technical Problem

However, in non-laparotomy using an endoscope, for example, since an object such as an organ cannot be directly touched with a hand, it is impossible to know hardness of the object as in laparotomy.

Thus, the present disclosure proposes an in-vivo observation system, an observation system, an in-vivo observation method, and an in-vivo observation device capable of quickly and robustly measuring hardness and a state of an object.

### Solution to Problem

According to the present disclosure, there is provided an in-vivo observation system including: an excitation device that vibrates an object in a living body; an event vision sensor that detects, as an event, a change due to the vibration in a luminance value of light emitted from the object; and an estimation unit that estimates a characteristic of the object on a basis of sensing data from the event vision sensor.

Furthermore, according to the present disclosure, there is provided an in-vivo observation system including: an excitation device that vibrates an object in a living body; an event vision sensor that detects, as an event, a change due to the vibration in a luminance value of light emitted from the object; and an estimation unit that estimates presence or absence of contact between the object and the excitation device on a basis of sensing data from the event vision sensor.

Furthermore, according to the present disclosure, there is provided an observation system including: an excitation device that vibrates an object; an event vision sensor that detects, as an event, a change due to the vibration in a luminance value of light emitted from the object; and an estimation unit that estimates a characteristic of the object on a basis of sensing data from the event vision sensor.

Furthermore, according to the present disclosure, there is provided an in-vivo observation method including: vibrating an object in a living body by using an excitation device; detecting a change due to the vibration in a luminance value of light, which is emitted from the object, as an event by using an event vision sensor; and estimating, by a computer, a characteristic of the object on a basis of sensing data from the event vision sensor.

Furthermore, according to the present disclosure, there is provided an in-vivo observation device including: an excitation device that vibrates an object in a living body; an event vision sensor that detects, as an event, a change due to the vibration in a luminance value of light emitted from the object; and an estimation unit that estimates a characteristic of the object on a basis of sensing data from the event vision sensor.

### BRIEF DESCRIPTION OF DRAWINGS

FIG. 1 is a view illustrating an example of a schematic configuration of an endoscopic surgery system to which a technology according to the present disclosure can be applied.

FIG. 2 is a description view for describing an outline of an embodiment of the present disclosure.

FIG. 3 is a block diagram illustrating an example of a configuration of an EVS 200 used in the embodiment of the present disclosure.

FIG. 4 is a block diagram illustrating an example of a configuration of a pixel 302 located in a pixel array unit 300 in the EVS 200 illustrated in FIG. 3.

FIG. 5 is a view illustrating an example of a configuration of a medical observation system 10 according to a first embodiment of the present disclosure.

FIG. 6 is a description view for describing an example of configurations of a camera head 100 and an optical system 400 illustrated in FIG. 5.

FIG. 7 is a description view for describing another example of the camera head 100 in the first embodiment of the present disclosure.

FIG. 8 is a description view (part 1) for describing a pattern projected from a light source device 600 onto a subject 950 in the first embodiment of the present disclosure.

FIG. 9 is a description view (part 2) for describing a pattern projected from the light source device 600 onto the subject 950 in the first embodiment of the present disclosure.

FIG. 10 is a description view for describing an irradiation pattern of the light source device 600 in the first embodiment of the present disclosure.

FIG. 11 is a description view for describing an example of an excitation device 150 in the first embodiment of the present disclosure.

FIG. 12 is a block diagram illustrating an example of a functional block configuration of a CCU 500 according to the first embodiment of the present disclosure.

FIG. 13 is a flowchart of a processing method according to the first embodiment of the present disclosure.

FIG. 14 is a description view for describing an example of a display in the first embodiment of the present disclosure.

FIG. 15 is a block diagram illustrating an example of a functional block configuration of a CCU 500a according to a second embodiment of the present disclosure.

FIG. 16 is a flowchart of a processing method according to the second embodiment of the present disclosure.

FIG. 17 is a hardware configuration diagram illustrating an example of a computer that realizes a CCU 500 according to an embodiment of the present disclosure.

### DESCRIPTION OF EMBODIMENTS

In the following, preferred embodiments of the present disclosure will be described in detail with reference to the accompanying drawings. Note that the same reference signs are assigned to components having substantially the same functional configuration, and overlapped description is omitted in the present specification and the drawings. In addition, in the present specification and the drawings, a plurality of components having substantially the same or similar functional configurations may be distinguished by assignment of different alphabets after the same reference sign. However, in a case where it is not specifically necessary to distinguish the plurality of components having substantially the same or similar functional configurations from each other, only the same reference sign is assigned.

Note that the description will be made in the following order.
1. Configuration example of an endoscopic surgery system 5000
1.1 Schematic configuration of the endoscopic surgery system 5000
1.2 Detailed configuration example of a support arm device 5027
1.3 Detailed configuration example of a light source device 5043
2. Background to creation of an embodiment of the present disclosure
2.1 Background to creation of the embodiment of the present disclosure
2.2 Outline of the embodiment of the present disclosure
2.3 About an EVS 200
3. First Embodiment
3.1 Configuration example of a medical observation system 10
3.2 Configuration example of a camera head 100 and an optical system 400
3.3 About a light source device 600
3.4 About an excitation device 150
3.5 Configuration example of a CCU 500
3.6 Processing method
4. Second Embodiment
4.1 Configuration example of a CCU 500a
4.2 Processing method
5. Conclusion
6. Hardware configuration
7. Supplementary note

### 1. Configuration Example of an Endoscopic Surgery System 5000

### 1.1 Schematic Configuration of the Endoscopic Surgery System 5000

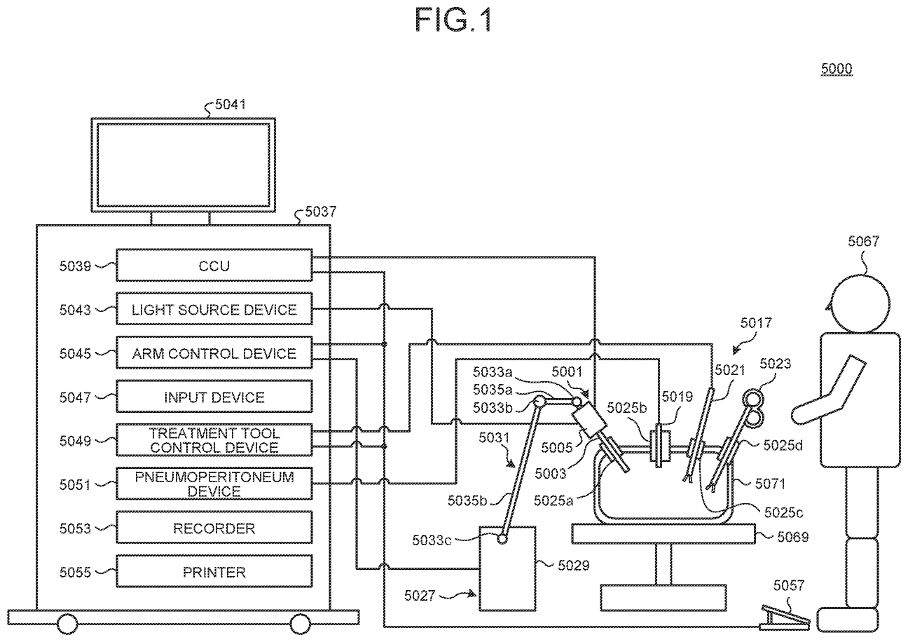

First, before describing details of an embodiment of the present disclosure, a schematic configuration of an endoscopic surgery system 5000 to which a technology according to the present disclosure can be applied will be described with reference to FIG. 1. FIG. 1 is a view illustrating an example of the schematic configuration of the endoscopic surgery system 5000 to which the technology according to the present disclosure can be applied. A state in which a surgeon 5067 performs surgery on a patient 5071 on a patient bed 5069 by using the endoscopic surgery system 5000 is illustrated in FIG. 1. As illustrated in FIG. 1, the endoscopic surgery system 5000 includes an endoscope 5001, another surgical tool 5017, a support arm device 5027 that supports the endoscope 5001, and a cart 5037 on which various devices for endoscopic surgery are mounted. Hereinafter, details of the endoscopic surgery system 5000 will be sequentially described.

(Surgical Tool 5017)

In endoscopic surgery, instead of cutting an abdominal wall and opening an abdomen, for example, a plurality of cylindrical puncture instruments called trocars 5025a to 5025d is punctured into the abdominal wall. Then, a lens barrel 5003 of the endoscope 5001 and the other surgical tool 5017 are inserted into a body cavity of the patient 5071 from the trocars 5025a to 5025d. In the example illustrated in FIG. 1, as the other surgical tools 5017, a pneumoperitoneum tube 5019, an energy treatment tool 5021, and forceps 5023 are inserted into the body cavity of the patient 5071. Furthermore, the energy treatment tool 5021 is a treatment tool that performs incision and separation of tissue, sealing of a blood vessel, or the like by a high-frequency current or ultrasonic vibration. However, the surgical tools 5017 illustrated in FIG. 1 are merely an example, and examples of the surgical tools 5017 include various surgical tools generally used in the endoscopic surgery, such as tweezers and a retractor.

(Support Arm Device 5027)

The support arm device 5027 includes an arm portion 5031 extending from a base portion 5029. In the example illustrated in FIG. 1, the arm portion 5031 includes joint portions 5033a, 5033b, and 5033c and links 5035a and 5035b, and is driven by control from an arm control device 5045. Then, the endoscope 5001 is supported by the arm portion 5031, and a position and posture of the endoscope 5001 are controlled. As a result, stable fixation of the position of the endoscope 5001 can be realized.

(Endoscope 5001)

The endoscope 5001 includes a lens barrel 5003 in which a region of a predetermined length from a distal end is inserted into the body cavity of the patient 5071, and a camera head 5005 connected to a proximal end of the lens barrel 5003. In the example illustrated in FIG. 1, the endoscope 5001 configured as a so-called rigid scope having a rigid lens barrel 5003 is illustrated. However, the endoscope 5001 may be configured as a so-called flexible scope having a flexible lens barrel 5003, and an embodiment of the present disclosure is not specifically limited.

An opening portion into which an objective lens is fitted is provided at the distal end of the lens barrel 5003. A light source device (medical light source device) 5043 is connected to the endoscope 5001, and light generated by the light source device 5043 is guided to the distal end of the lens barrel by a light guide extending inside the lens barrel 5003 and is emitted toward an observation target in the body cavity (such as in an abdominal cavity) of the patient 5071 via the objective lens. Note that in the embodiment of the present disclosure, the endoscope 5001 may be a forward-viewing endoscope or a forward-oblique viewing endoscope, and is not specifically limited.

An optical system and an imaging element are provided inside the camera head 5005, and radiation light (observation light) from the observation target is collected on the imaging element by the optical system. The observation light is photoelectrically converted by the imaging element, and an electric signal corresponding to the observation light, that is, a pixel signal corresponding to an observation image is generated. The pixel signal is transmitted to a camera control unit (CCU) 5039 as RAW data. Note that the camera head 5005 has a function of adjusting magnification and a focal length by appropriately driving the optical system.

Note that a plurality of various image sensors (not illustrated) may be provided in the camera head 5005, for example, in order to be compliant with stereoscopic viewing (stereoscopic system) or the like. In this case, a plurality of systems of relay optical systems and prisms may be provided inside the lens barrel 5003 in order to guide the observation light to each of the plurality of image sensors. Furthermore, different types of image sensors can be provided in the embodiment of the present disclosure, and a description thereof will be made later. Furthermore, details of the camera head 5005 and the lens barrel 5003 according to the embodiment of the present disclosure will also be described later.

(Various Devices Mounted on a Cart)

First, under the control of the CCU 5039, a display device 5041 displays an image based on a pixel signal on which image processing is performed by the CCU 5039. For example, in a case where the endoscope 5001 is compliant with high-resolution photographing such as 4K (the number of horizontal pixels 3840×the number of vertical pixels 2160) or 8K (the number of horizontal pixels 7680×the number of vertical pixels 4320), and/or in a case where the endoscope 5001 is compliant with a 3D display, a device capable of a high-resolution display and/or a device capable of a 3D display, which are respectively compliant with these cases are/is used as the display device 5041. Furthermore, a plurality of display devices 5041 having different kinds of resolution and different sizes may be provided depending on uses.

Furthermore, an image of a surgical site in the body cavity of the patient 5071 which image is captured by the endoscope 5001 is displayed on the display device 5041. While viewing the image of the surgical site displayed on the display device 5041 in real time, the surgeon 5067 can perform treatment such as resection of an affected part by using the energy treatment tool 5021 and the forceps 5023. Note that although not illustrated, the pneumoperitoneum tube 5019, the energy treatment tool 5021, and the forceps 5023 may be supported by the surgeon 5067, an assistant, or the like during surgery.

Furthermore, the CCU 5039 includes a central processing unit (CPU), a graphics processing unit (GPU), and the like, and can integrally control operation of the endoscope 5001 and the display device 5041. Specifically, the CCU 5039 performs, on the signal received from the camera head 5005, various kinds of image processing for displaying an image based on the signal, such as development processing (demosaic processing), for example. Furthermore, the CCU 5039 provides the signal on which the image processing is performed to the display device 5041. Furthermore, the CCU 5039 transmits a control signal to the camera head 5005 and controls driving thereof. The control signal can include information related to imaging conditions such as magnification and a focal length. Note that details of the CCU 5039 according to the embodiment of the present disclosure will be described later.

The light source device 5043 includes, for example, a light source such as a light emitting diode (LED), and supplies irradiation light in photographing of the surgical site to the endoscope 5001. Note that details of the light source device 5043 according to the embodiment of the present disclosure will be described later.

The arm control device 5045 includes, for example, a processor such as a CPU, operates according to a predetermined program, and controls driving of the arm portion 5031 of the support arm device 5027 according to a predetermined control method.

An input device 5047 is an input interface for the endoscopic surgery system 5000. The surgeon 5067 can input various kinds of information and instructions to the endoscopic surgery system 5000 via the input device 5047. For example, via the input device 5047, the surgeon 5067 inputs various kinds of information related to surgery, such as physical information of a patient and information related to a surgical procedure of the surgery. Furthermore, for example, via the input device 5047, the surgeon 5067 can input an instruction to drive the arm portion 5031, an instruction to change imaging conditions by the endoscope 5001 (type of irradiation light, magnification, focal length, and the like), an instruction to drive the energy treatment tool 5021, and the like. Note that a type of the input device 5047 is not limited, and the input device 5047 may be any of various known input devices. As the input device 5047, for example, a mouse, a keyboard, a touch panel, a switch, a foot switch 5057, a lever, and/or the like can be applied. For example, in a case where the touch panel is used as the input device 5047, the touch panel may be provided on a display surface of the display device 5041.

Alternatively, the input device 5047 may be a device worn on a part of a body of the surgeon 5067, such as a glasses-type wearable device or a head mounted display (HMD). In this case, various inputs are performed according to a gesture or a line of sight of the surgeon 5067, which is detected by these devices. Furthermore, the input device 5047 can include a camera capable of detecting a movement of the surgeon 5067, and various inputs may be performed according to a gesture or a line of sight of the surgeon 5067, which is detected from an image captured by the camera. Furthermore, the input device 5047 can include a microphone capable of collecting a voice of the surgeon 5067, and various inputs may be performed by the voice via the microphone. As described above, the input device 5047 is configured to be able to input various kinds of information in a non-contact manner. Thus, specifically, a user belonging to a clean area (such as the surgeon 5067) can operate a device belonging to an unclean area in the non-contact manner. In addition, since the surgeon 5067 can operate instrument without releasing his/her hand from the held surgical tool, convenience for the surgeon 5067 is improved.

A treatment tool control device 5049 controls driving of the energy treatment tool 5021 for cauterization or incision of tissue, sealing of a blood vessel, or the like. A pneumoperitoneum device 5051 feeds gas into the body cavity of the patient 5071 via a pneumoperitoneum tube 5019 in order to inflate the body cavity for a purpose of securing a visual field of the endoscope 5001 and securing a working space of the surgeon 5067. A recorder 5053 is a device capable of recording various kinds of information related to the surgery. The printer 5055 is a device capable of printing the various kinds of information related to the surgery in various formats such as a text, an image, or a graph.

### 1.2 Detailed Configuration Example of a Support Arm Device 5027

Furthermore, an example of a detailed configuration of the support arm device 5027 will be described. The support arm device 5027 includes the base portion 5029 that is a base, and the arm portion 5031 extending from the base portion 5029. In the example illustrated in FIG. 1, the arm portion 5031 includes the plurality of joint portions 5033a, 5033b, and 5033c and the plurality of links 5035a and 5035b coupled by the joint portion 5033b. However, in FIG. 1, the configuration of the arm portion 5031 is illustrated in a simplified manner for the sake of simplicity. Specifically, shapes, the number, and arrangements of the joint portions 5033a to 5033c and the links 5035a and 5035b, directions of rotation axes of the joint portions 5033a to 5033c, and the like can be appropriately set in such a manner that the arm portion 5031 has a desired degree of freedom. For example, the arm portion 5031 can be suitably configured to have 6 degrees of freedom or more. As a result, since the endoscope 5001 can be freely moved within a movable range of the arm portion 5031, the lens barrel 5003 of the endoscope 5001 can be inserted into the body cavity of the patient 5071 in a desired direction.

Actuators are provided in the joint portions 5033a to 5033c, and the joint portions 5033a to 5033c are configured to be rotatable around a predetermined rotation axis by driving of the actuators. The driving of the actuators is controlled by the arm control device 5045, whereby a rotation angle of each of the joint portions 5033a to 5033c is controlled, and the driving of the arm portion 5031 is controlled. As a result, control of a position and posture of the endoscope 5001 can be realized. At this time, the arm control device 5045 can control the driving of the arm portion 5031 by various known control methods such as force control or position control.

For example, when the surgeon 5067 appropriately performs an operation input via the input device 5047 (including the foot switch 5057), the driving of the arm portion 5031 may be appropriately controlled by the arm control device 5045 according to the operation input, and the position and posture of the endoscope 5001 may be controlled. Note that the arm portion 5031 may be operated by a so-called master-slave method. In this case, the arm portion 5031 (slave) (such as an arm included in a payment cart) can be remotely controlled by the surgeon 5067 via the input device 5047 (master console) installed at a place remote from an operating room or in the operating room.

Here, in general, in endoscopic surgery, the endoscope 5001 is supported by a doctor called scopist. On the other hand, in the embodiment of the present disclosure, since the position of the endoscope 5001 can be more reliably fixed by utilization of the support arm device 5027 without manual operation, an image of the surgical site can be stably acquired, and surgery can be smoothly performed.

Note that the arm control device 5045 is not necessarily provided in the cart 5037. Furthermore, the arm control device 5045 is not necessarily one device. For example, the arm control device 5045 may be provided in each of the joint portions 5033a to 5033c of the arm portion 5031 of the support arm device 5027, and the drive control of the arm portion 5031 may be realized by the plurality of arm control devices 5045 cooperating with each other.

### 1.3 Detailed Configuration Example of a Light Source Device 5043

Next, an example of the detailed configuration of the light source device 5043 will be described. The light source device 5043 supplies the endoscope 5001 with irradiation light in photographing of the surgical site. The light source device 5043 includes, for example, an LED, a laser light source, or a white light source including a combination thereof. At this time, in a case where the white light source includes a combination of RGB laser light sources, output intensity and output timing of each color (each wavelength) can be controlled with high accuracy, whereby a white balance of the captured image can be adjusted in the light source device 5043. Furthermore, in this case, by emitting laser light from each of the RGB laser light sources to the observation target in a time division manner and controlling driving of the imaging element of the camera head 5005 in synchronization with the emission timing, it is also possible to capture an image corresponding to each of RGB in a time division manner. According to the method, a color image can be acquired even when a color filter is not included in the imaging element.

Furthermore, driving of the light source device 5043 may be controlled in such a manner that intensity of output light is changed every predetermined time. By controlling the driving of the imaging element of the camera head 5005 in synchronization with timing of the change in the intensity of the light, acquiring images in a time division manner, and synthesizing the images, it is possible to generate an image of a high dynamic range without so-called blocked up shadows and blown out highlights.

Furthermore, the light source device 5043 may be configured to be able to supply light in a predetermined wavelength band corresponding to special light observation. In the special light observation, for example, so-called narrow band imaging in which a predetermined tissue such as a blood vessel in a superficial portion of the mucous membrane is photographed with high contrast by emission of light in a narrower band than irradiation light at the time of normal observation (that is, white light) by utilization of wavelength dependency of light absorption in a body tissue is performed. Alternatively, in the special light observation, fluorescence observation for acquiring an image by fluorescence generated by emission of excitation light may be performed. In the fluorescence observation, for example, observation in which excitation light is emitted to a body tissue and fluorescence from the body tissue is observed (autofluorescence observation), or observation in which a fluorescent image is acquired by local injection of a reagent such as indocyanine green (ICG) into a body tissue and emission of excitation light corresponding to a fluorescence wavelength of the reagent to the body tissue may be performed. The light source device 5043 can be configured to be able to supply narrow band light and/or excitation light corresponding to such special light observation. Furthermore, in the embodiment of the present disclosure, the light source device 5043 can project light having a pattern on the observation target. Note that details of the light source device 5043 will be described later.

### 2. Background to Creation of an Embodiment of the Present Disclosure

### 2.1 Background to Creation of the Embodiment of the Present Disclosure

In recent years, in a surgical operation, by progress of peripheral technologies of the endoscope 5001, such as the endoscope 5001 and the endoscopic surgery system 5000 described above, it has become possible to realize minimally invasive non-laparotomy in which a wound is small and recovery after the surgery is fast as compared with laparotomy. However, on the other hand, in the non-laparotomy using the endoscope 5001, for example, an object such as an organ cannot be directly touched by a hand, whereby it is impossible to know hardness of the object as in the laparotomy. In the surgical operation or the like, hardness information of the organ is, for example, a guide of a tension state of a portion around anastomosis at the time of an anastomosis operation of the organ, and is important information for preventing a complication due to an anastomosis failure or a blood circulation failure. In addition, the hardness information of the organ can also be important information when a hardened part of a tissue due to cancer or the like is checked. Thus, there has been a strong demand for a method for knowing hardness of an organ which method can be used in non-laparotomy or the like using the endoscope 5001.

As a method for knowing hardness of an object in a non-contact manner, the following method already exists. For example, an ultrasonic wave or laser light is emitted to the object, the object is vibrated, displacement caused by the vibration, that is, amplitude is measured, and the measured amplitude is applied to a model of viscoelastic impedance, whereby the hardness of the object can be estimated. However, in such a method, the ultrasonic wave or the laser light is converged on the object and scanning is performed, whereby hardness distribution in the entire object or a wide range of the object is measured. Thus, it takes time to measure the hardness in the wide region. In addition, in a case where the object is an organ, considering a size and the like thereof and an influence on a human body, it is not possible to vibrate the object greatly or vibrate the object for a long time. Thus, displacement due to vibration (amplitude) becomes minute and fast, and it is difficult to accurately perform measurement thereof. Furthermore, in such a method, since measurement time is long, a measurement error is likely to be generated due to movement of a position itself of the organ or movement of a hand of the scopist during the measurement.

### 2.2 Outline of the Embodiment of the Present Disclosure

Then, in view of the above-described situation, the present inventors have intensively studied, for example, a method of quickly and robustly measuring hardness of an object such as an organ. During such studies, the present inventors have reached an idea that a method of quickly and robustly measuring the hardness of the object such as the organ can be realized by utilization of an event vision sensor (EVS).

The EVS is an image sensor that sensitively detects a luminance change, and has higher sensitivity than a general RGB sensor. In addition, the EVS does not have a concept of a frame rate, and can output time stamp information and pixel information when a generated luminance change exceeds a threshold. Thus, the EVS can output information at a high frame rate according to frequent luminance changes, in other words, minute displacement of a desired object can be captured with high time resolution.

Thus, the present inventors have uniquely created a method of estimating hardness of an object by applying vibration to an object (such as an organ) (excitation) and capturing minute and high-speed displacement (deformation) generated by the vibration with high time resolution by the above-described EVS.

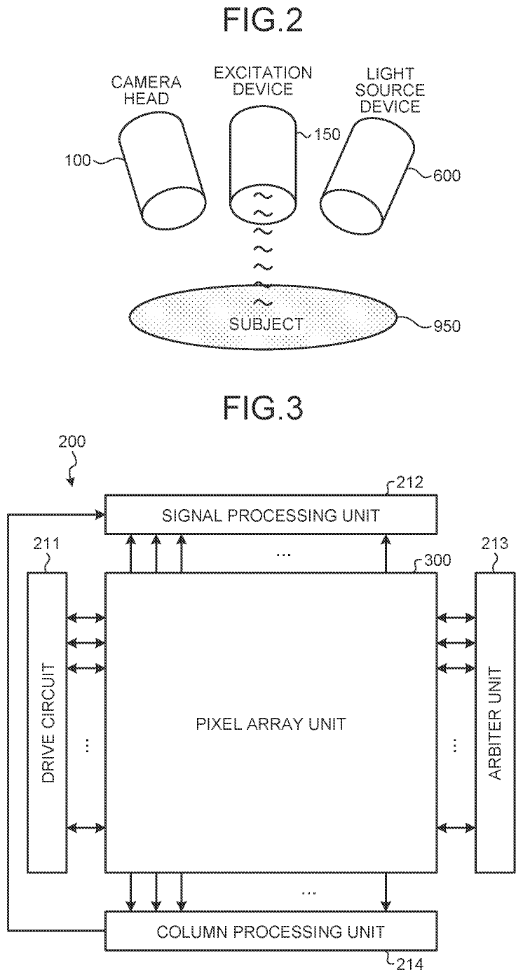

Specifically, in the embodiment of the present disclosure created by the present inventors, first, vibration is applied to a subject (object) 950 such as an organ by the excitation device 150 as illustrated in FIG. 2 that is a description view for describing an outline of the embodiment of the present disclosure. Then, in the embodiment of the present disclosure, displacement of the subject 950 irradiated with light (such as light having a pattern) by the light source device 600 is captured by the camera head 100 including the EVS described above, and hardness of the subject 950 is estimated from the captured vibration state.

In the embodiment of the present disclosure created by the present inventors, minute and high-speed displacement of the subject 950 vibrated by the excitation device 150 can be captured by EVS having high time resolution. Thus, according to the embodiment of the present disclosure, hardness of an object such as an organ can be measured quickly and robustly. Hereinafter, details of such an embodiment of the present disclosure will be sequentially described.

### 2.3 About an EVS 200

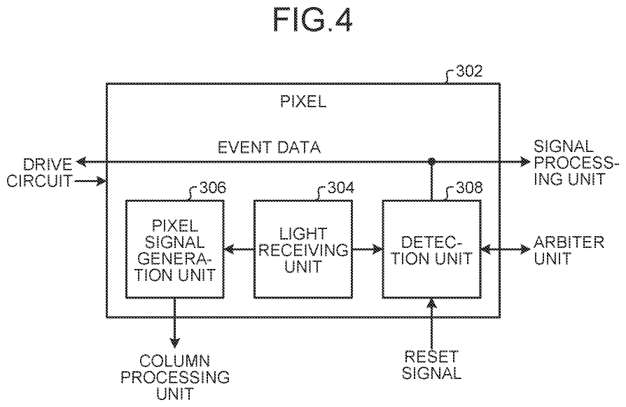

Here, the EVS will be described with reference to FIG. 3 and FIG. 4. FIG. 3 is a block diagram illustrating an example of a configuration of the EVS 200 used in the embodiment of the present disclosure, and FIG. 4 is a block diagram illustrating an example of a configuration of the pixels 302 located in a pixel array unit 300 in the EVS 200 illustrated in FIG. 3.

As illustrated in FIG. 3, the EVS 200 includes the pixel array unit 300 configured by an array of the plurality of pixels 302 (see FIG. 4) in a matrix. Each of the pixels 302 can generate, as a pixel signal, a voltage corresponding to a photocurrent generated by photoelectric conversion. Furthermore, each of the pixels 302 can detect presence or absence of an event by comparing a change in the photocurrent corresponding to a luminance change amount of incident light (radiation light from an object) with a predetermined threshold. In other words, the pixel 302 can detect an event on the basis of the fact that the luminance change amount exceeds the predetermined threshold.

Furthermore, as illustrated in FIG. 3, the EVS 200 includes a drive circuit 211, an arbiter unit (arbitration unit) 213, a column processing unit 214, and a signal processing unit 212 as peripheral circuit units of the pixel array unit 300.

When detecting an event, each of the pixels 302 can output, to the arbiter unit 213, a request for requesting an output of event data indicating generation of the event. Then, in a case of receiving a response indicating permission for the output of the event data from the arbiter unit 213, each of the pixels 302 outputs the event data to the drive circuit 211 and the signal processing unit 212. Furthermore, the pixel 302 that detects the event outputs a pixel signal generated by photoelectric conversion to the column processing unit 214.

The drive circuit 211 can drive each of the pixels 302 of the pixel array unit 300. For example, the drive circuit 211 drives the pixel 302 that detects an event and that outputs event data, and causes a pixel signal of the corresponding pixel 302 to be output to the column processing unit 214.

The arbiter unit 213 can arbitrate the request for requesting the output of the event data which request is supplied from each of the pixels 302, and can transmit, to the pixel 302, a response based on a result of the arbitration (permission/non-permission for the output of the event data) and a reset signal for resetting the event detection.

For each column of the pixel array unit 300, the column processing unit 214 can perform processing of converting analog pixel signals output from the pixels 302 in the corresponding column into digital signals. The column processing unit 214 can also perform correlated double sampling (CDS) processing on the digitized pixel signals.

The signal processing unit 212 can execute predetermined signal processing on the digitized pixel signals supplied from the column processing unit 214 and the event data output from the pixel array unit 300, and can output the event data (such as time stamp information) and the pixel signals after the signal processing.

The change in the photocurrent generated in each of the pixels 302 can be regarded as a light quantity change (luminance change) in light incident on the pixel 302. Thus, it can also be said that the event is a luminance change in the pixel 302 which change exceeds the predetermined threshold. Furthermore, the event data indicating the generation of the event can include at least position information such as coordinates indicating a position of the pixel 302 in which the light quantity change as the event is generated.

Furthermore, the pixels 302 will be described with reference to FIG. 4. In the pixel array unit 300 configured by an array of the plurality of pixels 302 in a matrix, each of the pixels 302 includes a light receiving unit 304, a pixel signal generation unit 306, and a detection unit (event detection unit) 308.

Specifically, the light receiving unit 304 can photoelectrically convert incident light and generates a photocurrent. Then, the light receiving unit 304 can supply a signal of a voltage corresponding to the photocurrent to either the pixel signal generation unit 306 or the detection unit 308 under the control of the drive circuit 211.

The pixel signal generation unit 306 can generate, as a pixel signal, the signal supplied from the light receiving unit 304. Then, the pixel signal generation unit 306 can supply the generated analog pixel signal to the column processing unit 214 via a vertical signal line VSL (not illustrated) corresponding to a column of the pixel array unit 300.

The detection unit 308 can detect whether an event is generated on the basis of whether the change amount of the photocurrent from the light receiving unit 304 exceeds a predetermined threshold. The event can include, for example, an on-event indicating that the change amount of the photocurrent (luminance change amount) exceeds an upper limit threshold, and an off-event indicating that the change amount thereof falls below a lower limit threshold. Note that the detection unit 308 may detect only the on-event.

When the event is generated, the detection unit 308 can output, to the arbiter unit 213, a request for requesting the output of event data indicating the generation of the event. Then, in a case of receiving a response to the request from the arbiter unit 213, the detection unit 308 can output the event data to the drive circuit 211 and the signal processing unit 212.

In the embodiment of the present disclosure, by applying such an EVS 200, it is possible to utilize high robustness and high time resolution in detection of a fast moving subject, which are characteristics of the EVS 200. Thus, it is possible to accurately capture the minute and high-speed displacement of the subject 950 vibrated by the excitation device 150.

### 3. First Embodiment

### 3.1 Configuration Example of a Medical Observation System 10

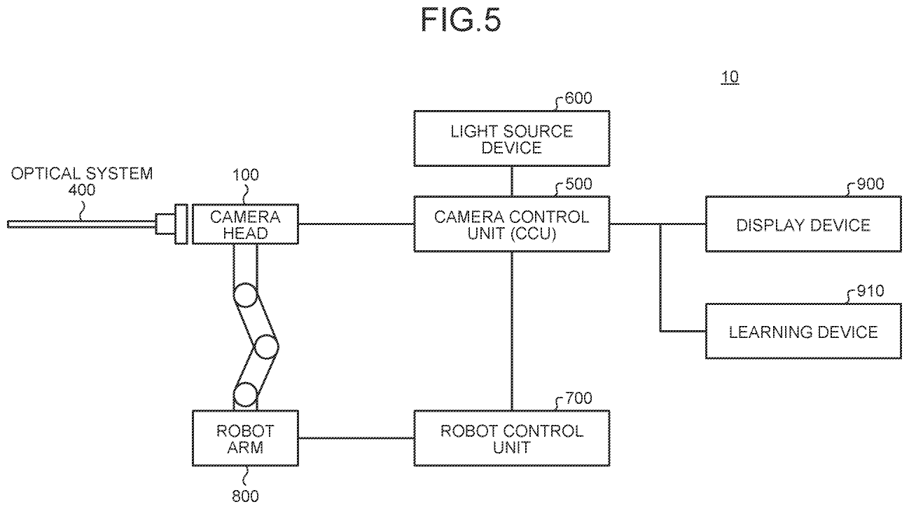

First, a configuration example of the medical observation system (in-vivo observation system and in-vivo observation device) 10 according to the first embodiment of the present disclosure will be described with reference to FIG. 5. FIG. 5 is a view illustrating an example of a configuration of the medical observation system 10 according to the present embodiment. The medical observation system 10 can be applied to the endoscopic surgery system 5000 described above.

As illustrated in FIG. 5, the medical observation system 10 mainly includes a camera head 100 (corresponding to the camera head 5005 described above), an optical system 400 (corresponding to the lens barrel 5003 described above), a camera control unit (CCU) (information processing device) 500 (corresponding to the CCU 5039 described above), a light source device 600 (corresponding to the light source device 5043 described above), a robot control unit 700 (corresponding to the arm control device 5045 described above), a robot arm 800 (corresponding to the support arm device 5027 described above), a display device 900 (corresponding to the display device 5041 described above), and a learning device 910. Hereinafter, each device included in the medical observation system 10 will be described.

First, before description of details of the configuration of the medical observation system 10, an outline of an operation of the medical observation system 10 will be described. In the medical observation system 10, by controlling the robot arm 800 by using the robot control unit 700, it is possible to fix positions of the camera head 100 and the optical system 400, which are supported by the robot arm 800, at suitable positions without manual operation. Thus, according to the medical observation system 10, since the image of the surgical site can be stably obtained, the surgeon 5067 can smoothly perform the surgery. Note that in the following description, a person who moves or fixes a position of the endoscope 5001 is referred to as a scopist, and the operation of the endoscope 5001 (including a movement, a stop, a change in posture, zoom-in, zoom-out, and the like) is referred to as a scope work regardless of manual operation or mechanical control.

(Camera Head 100 and Optical System 400)

The camera head 100 and the optical system 400 are provided at a distal end of the robot arm 800 (described later), and capture an image of a subject (such as intraperitoneal environment) 950 that is an object of various kinds of imaging. In other words, the robot arm 800 supports the camera head 100 and the optical system 400. Note that the camera head 100 and the optical system 400 may be, for example, a forward-oblique viewing endoscope, a forward-viewing endoscope with a wide-angle/cutout function (not illustrated), an endoscope with a distal end bending function (not illustrated), an endoscope with a simultaneous photographing function in another direction (not illustrated), or an exoscope or a microscope, and are not specifically limited.

Furthermore, the camera head 100 and the optical system 400 can include, for example, an image sensor (not illustrated) that can capture an operative field image (observation image) including various surgical tools in an abdominal cavity of a patient, an organ (object in the living body), and the like, the EVS 200 described above, and the like. Specifically, the camera head 100 can function as a camera capable of photographing a photographing target in a form of a moving image or a still image. Furthermore, the camera head 100 can transmit an electric signal (pixel signal) corresponding to the captured image to the CCU 500 (described later). Note that the robot arm 800 may support the excitation device 150 that vibrates a surgical tool such as the forceps 5023, an organ, or the like. Furthermore, the excitation device 150 may be provided at a distal end portion of the camera head 100 or the optical system 400.

Furthermore, in the present embodiment, the camera head 100 and the optical system 400 may be stereoscopic endoscopes capable of performing ranging. Alternatively, a depth sensor (ranging device) (not illustrated) may be provided in the camera head 100 or separately from the camera head 100. The depth sensor can be, for example, a sensor that performs ranging by using a time of flight (ToF) method in which ranging is performed by utilization of a return time of reflection of pulsed light from the subject 950, or a structured light method in which lattice-shaped pattern light is emitted and ranging is performed according to distortion of the pattern.

Note that details of the camera head 100 and the optical system 400 in the present embodiment (specifically, the RGB sensor, the EVS 200, the excitation device 150, and the like) will be described later.

(CCU 500)

As described above, the CCU 500 includes a CPU, a GPU, or the like, and can integrally control the operation of the camera head 100. Furthermore, the CCU 500 can perform various kinds of image processing for displaying an image on the pixel signal (sensing data) received from the camera head 100, and analyze the image. Furthermore, the CCU 500 provides the pixel signal, on which the image processing is performed, to the display device 900 (described later). Furthermore, the CCU 500 can transmit a control signal to the camera head 100 and control driving thereof. The control signal can include information related to imaging conditions such as magnification and a focal length. Note that details of the CCU 500 in the present embodiment will be described later.

(Light Source Device 600)

The light source device 600 emits light to the subject 950 in a living body, which is an imaging target of the camera head 100. The light source device 600 can be realized by, for example, an LED for a wide-angle lens. For example, the light source device 600 may be configured by a combination of a normal LED and a lens, and may diffuse light. Furthermore, the light source device 600 may have a configuration in which light transmitted through an optical fiber (light guide) is diffused (widened) by a lens, for example. In addition, the light source device 600 may expand an irradiation range by emitting light while directing the optical fiber itself in a plurality of directions. Note that details of the light source device 600 in the present embodiment will be described later.

(Robot Control Unit 700)

The robot control unit 700 controls driving of the robot arm 800 (described later). The robot control unit 700 is realized by, for example, a CPU, a micro processing unit (MPU), or the like that executes a program stored in a storage unit (described later) (such as a program according to an embodiment of the present disclosure) by using a random access memory (RAM) or the like as a work area. Also, the robot control unit 700 is a controller, and may be realized by an integrated circuit such as an application specific integrated circuit (ASIC) or a field programmable gate array (FPGA), for example. Furthermore, the robot control unit 700 may be a device integrated with the CCU 500 described above, or may be a separate device. Furthermore, in the present embodiment, for example, on the basis of data from the CCU 500 (such as hardness of the subject 950 and presence or absence of contact with the subject 950) (estimation result), the robot control unit 700 may control the robot arm 800 to autonomously operate.

(Robot Arm 800)

As described above, the robot arm 800 includes an articulated arm (corresponding to the arm portion 5031 illustrated in FIG. 1) that is a multilink structure including a plurality of joint units and a plurality of links. Then, by driving of the robot arm 800 within a movable range, positions and postures of the camera head 100 and the optical system 400 provided at the distal end of the robot arm 800 can be controlled. Furthermore, the robot arm 800 may include a motion sensor (not illustrated) including an acceleration sensor, a gyroscope sensor, a geomagnetic sensor, and the like in order to acquire data of a position and posture of the robot arm 800.

(Display Device 900)

The display device 900 displays various images. The display device 900 displays, for example, an image captured by the camera head 100. The display device 900 can be, for example, a display including a liquid crystal display (LCD), an organic electro-luminescence (EL) display, or the like. Note that the display device 900 may be a device integrated with the CCU 500 illustrated in FIG. 5 described above, or may be a separate device connected to the CCU 500 in a manner of being communicable therewith in a wired or wireless manner.

(Learning Device 910)

The learning device 910 includes, for example, a CPU, an MPU, or the like, and can perform machine learning by using, for example, an image (annotation) or the like captured by the camera head 100. The learning model acquired by such machine learning can be used for image diagnosis or the like. Furthermore, by using the image or the like acquired by the camera head 100, the learning device 910 can also generate a learning model used when autonomous operation control information for causing the robot arm 800 to autonomously operate is generated.

Note that in the present embodiment, the configuration of the medical observation system 10 is not limited to the configuration illustrated in FIG. 5, and may include another device or the like, and may not include the learning device 910 or the like.

### 3.2 Configuration Example of a Camera Head 100 and an Optical System 400

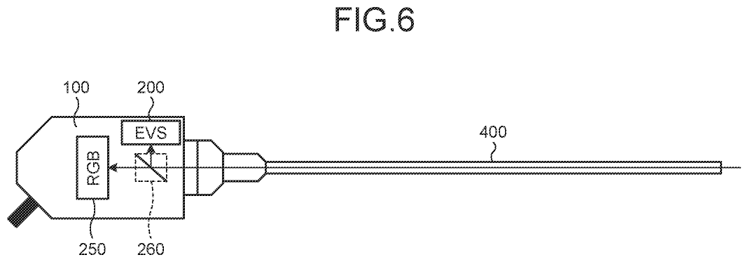

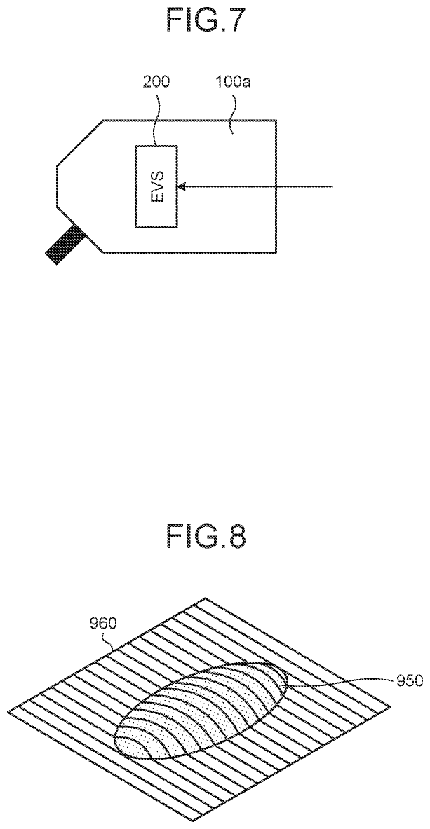

Next, an example of detailed configurations of the camera head (camera head unit) 100 and the optical system 400 according to the present embodiment will be described with reference to FIG. 6 and FIG. 7. FIG. 6 is a description view for describing an example of the configurations of the camera head 100 and the optical system 400 illustrated in FIG. 5, and FIG. 7 is a description view for describing another example of the configuration of the camera head 100 in the present embodiment.

(Camera Head 100)

Specifically, as illustrated in FIG. 6, the camera head 100 includes an EVS 200, an RGB sensor (image sensor) 250, and a prism 260. As described above, the EVS 200 can detect, as event data, that a luminance change amount caused by light incident on each of the plurality of pixels 302 arrayed in a matrix exceeds a predetermined threshold. More specifically, in the present embodiment, the EVS 200 can capture, as the event data, a change in the luminance value of the light from the subject 950 due to minute and high-speed displacement of the subject 950 vibrated by the excitation device 150. Then, the event data detected by the EVS 200 is transmitted to the CCU 500. Since the detailed configuration of the EVS 200 has been described above, the description thereof will be omitted here.

The RGB sensor 250 acquires radiation light from the subject 950 in order to acquire an observation image of the subject 950 based on the radiation light from the subject 950. Then, a pixel signal output from the RGB sensor 250 is transmitted to the CCU 500. Specifically, the RGB sensor 250 is, for example, an image sensor that has a Bayer array capable of detecting blue light, green light, and red light and that can perform color photographing, and is preferably, for example, an image sensor that can be compliant with photographing of a high-resolution image of 4K or more. By utilization of such an image sensor, an image of 950 such as an organ can be acquired with high resolution. Thus, the surgeon 5067 can grasp a state of a surgical site in more detail and can proceed the surgery more smoothly. Furthermore, the RGB sensor 250 may include a pair of image sensors for respectively acquiring right-eye and left-eye images corresponding to 3D display (stereoscopic system). By the 3D display, the surgeon 5067 can more accurately grasp a depth of the organ in the surgical site and grasp a distance to the organ.

The prism 260 can guide reflection light from the subject 950 to both the EVS 200 and the RGB sensor 250. Furthermore, the prism 260 may have a function of adjusting a distribution ratio of the quantity of light incident on each of the EVS 200 and the RGB sensor 250. For example, the above function can be provided by an adjustment of transmittance of the prism 260. More specifically, for example, in a case where optical axes of the incident light are the same between the EVS 200 and the RGB sensor 250, it is preferable to adjust the transmittance of the prism 260 in such a manner that the quantity of light incident on a side of the RGB sensor 250 becomes larger.

Note that in the present embodiment, the configuration is not limited to such a configuration in which the EVS 200 and the RGB sensor 250 are provided on different substrates and light is guided to both the EVS 200 and the RGB sensor 250 by the prism 260. In the present embodiment, for example, a hybrid-type sensor in which pixel arrays corresponding to the EVS 200 and the RGB sensor 250 are provided on the same substrate (light receiving surface) may be used. In such a case, the above-described prism 260 is unnecessary, and the configuration in the camera head 100 can be simplified.

Furthermore, in the present embodiment, two or three or more EVSs 200 and RGB sensors 250 may be provided in order to enable a stereoscopic system capable of perform ranging. Furthermore, in a case of realizing the stereoscopic system, two optical systems 400 may be made to correspond to one pixel array and two image circles may be projected on the one pixel array.

Furthermore, in the present embodiment, the camera head 100 may include an IR sensor (not illustrated) that detects infrared light, or may include a short wavelength infrared (SWIR) sensor such as an InGaAs sensor (not illustrated). For example, a blood vessel or the like at a deep position in the body can be accurately captured by utilization of short wavelength infrared light (light having a wavelength of about 900 nm to about 2500 nm).

Furthermore, in the present embodiment, the EVS 200 and the RGB sensor 250 may be provided not in the camera head 100 but at a distal end portion of a flexible endoscope or a rigid endoscope inserted into the abdominal cavity.

(Optical System 400)

The optical system 400 can guide radiation light from the subject 950 to the camera head 100. Specifically, the radiation light from the subject 950 is guided to the camera head 100 by an imaging optical system (not illustrated) included in the optical system 400, and is collected on the pixel array unit 300 of the EVS 200 (see FIG. 3). In addition, an imaging optical system 402 is configured by a combination of a plurality of lenses including a zoom lens and a focus lens. Furthermore, the zoom lens and the focus lens may be configured to be movable in a position on optical axes thereof for an adjustment of a magnification and a focus of a captured image, and the like.

Furthermore, in the present embodiment, the optical system 400 may include a light source optical system (not illustrated) that guides light from the light source device 600 to the subject 950. Furthermore, the light source optical system may be configured by a combination of a plurality of lenses including a zoom lens and a focus lens.

Furthermore, in the present embodiment, as illustrated in FIG. 7, only an EVS 200 may be provided in a camera head 100a. In addition, the EVS 200 and the RGB sensor 250 may be provided in different camera heads 100. In such a case, the camera head 100 provided with the EVS 200 and the camera head 100 provided with the RGB sensor 250 may be respectively supported by different robot arms 800.

Furthermore, in the present embodiment, the camera head 100, the optical system 400, and the like have a sealed structure with high airtightness and waterproofness, whereby the camera head 100 and the optical system 400 can have resistance to autoclave sterilization.

### 3.3 about a Light Source Device 600

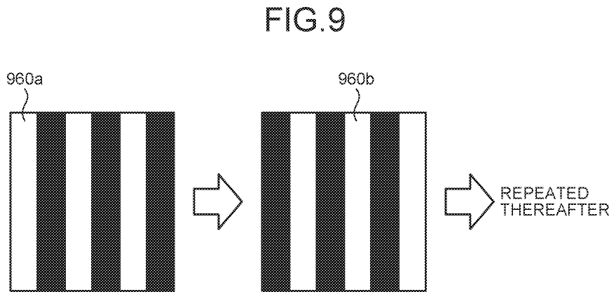

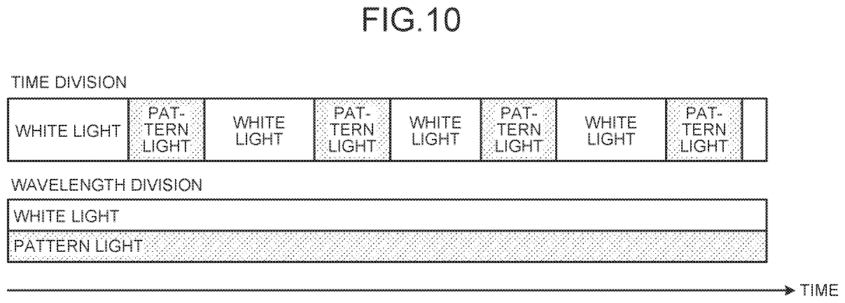

Next, details of the light source device 600 according to the present embodiment will be described with reference to FIG. 8 to FIG. 10. FIG. 8 and FIG. 9 are description views for describing a pattern projected from the light source device 600 onto the subject 950. FIG. 10 is a description view for describing an irradiation pattern of the light source device 600.

As described above, the light source device 600 includes, for example, a light source such as an LED, and emits light to the subject 950, which is an imaging target of the camera head 100, on the basis of a control signal from the CCU 500. In the present embodiment, the light source device 600 can emit, to the subject 950, light having a predetermined wavelength, such as red light, blue light, and green light in a visible light (having a wavelength of about 360 nm to about 830 nm) range, white light in which pieces of light of all wavelengths in the visible light range (red light, blue light, and green light) are uniformly mixed, infrared light (light having a wavelength of about 700 nm to about 1 mm), short-wavelength infrared light (having a wavelength of about 900 nm to about 2500 nm), and the like.

Furthermore, in the present embodiment, for example, as illustrated in FIG. 8, the light source device 600 can project light having a slit pattern (predetermined pattern) 960 onto the subject 950. In the present embodiment, light having such a slit pattern 960 is projected, and instead of displacement of the subject 950 vibrated by the excitation device 150, distortion of the slit pattern 960 due to the displacement is captured by the EVS 200. In such a manner, according to the present embodiment, since the minute displacement of the subject 950 is replaced with the distortion of the slit pattern 960 and captured, it is possible to easily capture the minute and high-speed displacement of the vibrating subject 950. Note that in the present embodiment, the pattern to be projected onto the subject 950 is not limited to the slit pattern 960, and may be a lattice pattern, a moire fringe pattern, or the like. Furthermore, in the present embodiment, a width and interval of the pattern are not specifically limited, and are preferably appropriately selected according to a size, shape, and the like of the subject 950.

Furthermore, in the present embodiment, for example, as illustrated in FIG. 9, the light source device 600 may repeatedly and continuously emit, to the subject 950, a slit pattern 960a and a slit pattern 960b having a mutually inverted pattern. In the present embodiment, by projecting light in such a manner, the minute and high-speed displacement of the subject 950 vibrated by the excitation device 150 can be easily captured.

Furthermore, in the present embodiment, the light source device 600 can emit, to the subject 950, light for the EVS 200 and light for the RGB sensor 250 by the emission with time division or wavelength division of the light. In the present embodiment, an organ or the like in the abdominal cavity is the subject 950. In such a case, since there is no external light, by performing the time division or the wavelength division as described above, the light source device 600 can emit, to the subject 950, light for the EVS 200 for identifying a characteristic of the subject 950 (first light) and light for the RGB sensor 250 for generating an image (observation image) of the subject 950 (second light).

Specifically, for example, as illustrated in an upper part of FIG. 10, the light source device 600 may emit pattern light having the slit pattern 960 for the EVS 200 (first light) and white light for the RGB sensor 250 (second light) alternately in time (time division). Note that in the present embodiment, since measurement time of the hardness of the subject 950 is short, the pattern light may be emitted for a shorter time than the white light. Furthermore, at the time of performing such light emission, the light source device 600, the camera head 100, and the excitation device 150 are controlled to be synchronized by the CCU 500.

Furthermore, in the present embodiment, as illustrated in a lower part of FIG. 10, for example, the light source device 600 may simultaneously emit, to the subject 950, pattern light having a wavelength of infrared light for the EVS 200 (first light) and white light for the RGB sensor 250 (second light) (wavelength division). That is, the light source device 600 can emit pieces of light having different wavelengths as the light for the EVS 200 and the light for the RGB sensor 250. Such wavelength division can be realized, for example, by utilization of a filter that transmits only light having a wavelength in a predetermined range.

Note that in the present embodiment, in a case where the light emitted from the light source device 600 has the slit pattern 960, it is preferable that the light source device 600 and the camera head 100 including the EVS 200 are coaxially positioned with respect to the subject 950. In addition, in the present embodiment, in a case where the light emitted from the light source device 600 does not has the slit pattern 960, the light source device 600 and the camera head 100 including the EVS 200 does not need to be coaxially positioned with respect to the subject 950.

### 3.4 about an Excitation Device 150

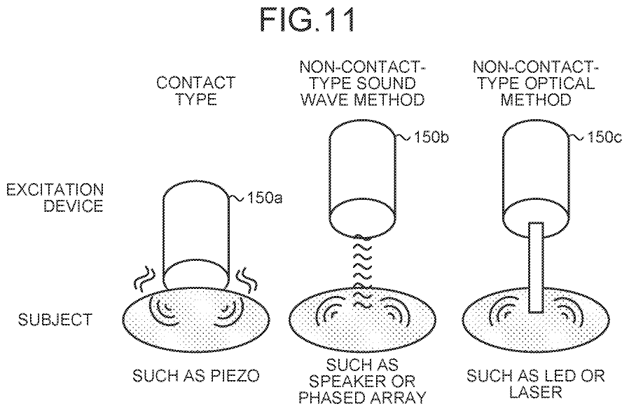

Next, details of the excitation device 150 according to the present embodiment will be described with reference to FIG. 11. FIG. 11 is a description view for describing an example of the excitation device 150.

The excitation device 150 that vibrates the subject 950 can be provided at a distal end portion of the robot arm 800, or a distal end portion of the camera head 100, the optical system 400, or the surgical tool supported by the robot arm 800. Furthermore, as illustrated in FIG. 11, the excitation device 150 can be mainly divided into two types that are a contact type that applies vibration in contact with the subject 950, and a non-contact type that applies vibration without contacting the subject 950.

Specifically, in the present embodiment, as illustrated on a left side of FIG. 11, a vibrator 150a such as a piezoelectric element that can perform vibration by voltage application can be used as the contact-type excitation device 150. Furthermore, in the present embodiment, an actuator including a motor and components may be used as the excitation device 150 in addition to the vibrator. In the present embodiment, such a vibrator or the like is provided at the distal end portion of the robot arm 800, the camera head 100, the optical system 400, or the surgical tool, and can directly contact the subject 950 such as the organ and apply vibration to the subject 950. According to the present embodiment, by using such a vibrator, it is possible to realize a configuration of exciting the subject 950 at low a cost without greatly changing the configuration of the medical observation system 10.

Furthermore, in the present embodiment, a device capable of performing excitation by a sound wave method or an optical method can be assumed as the non-contact-type excitation device 150 as illustrated at a center and on a right side of FIG. 11. Specifically, for example, in a case where the sound wave method is employed, a speaker, a phased array, or the like can be used as an excitation device 150b of the sound wave method, and the subject 950 can be excited by emission of a sound wave such as an ultrasonic wave to the subject 950. Furthermore, for example, in a case where the optical method is employed, by utilization of an LED, a laser, or the like as an excitation device 150c of the optical method, the subject 950 can be excited by emission of light to the subject 950. In the present embodiment, by using such an excitation device 150 of the sound wave method or the optical method, it is possible to realize the configuration of exciting the subject 950 without greatly changing the configuration of the medical observation system 10. Furthermore, in a case where the subject 950 is an affected part such as a blood vessel or an aneurysm, a shape or a state may be greatly changed by a direct contact on the affected part, or the state of the affected part may be deteriorated in some cases. In the present embodiment, by using the non-contact-type excitation device 150 as described above, excitation can be performed without contact on the affected part. Thus, the shape and state of the affected part are not changed.

Note that in the present embodiment, a distance between the non-contact-type excitation device 150 and the subject 950 is preferably adjusted according to a characteristic of the subject 950, a size of a range to be excited, and the like. Furthermore, in the present embodiment, it is preferable that a frequency (wavelength) of the sound wave or light emitted from the excitation device 150 to the subject 950 is also appropriately selected according to the characteristic of the subject 950, the size of the range to be excited, and the like. Furthermore, in the present embodiment, the frequency of the sound wave or the light may be swept (continuously changed) according to the characteristic of the subject 950, or the like. From the above, in the present embodiment, it is possible to observe a change in vibration based on an absorption characteristic of the subject 950 with respect to the sound wave and the light. Thus, it is possible to more accurately estimate hardness (hardness) of the subject 950.

Furthermore, in the present embodiment, the subject 950 may be continuously excited or may be excited intermittently (in a pulse form). It is preferable to appropriately select an excitation pattern according to the characteristic of the subject 950, the size of the range to be excited, a use, and the like. Furthermore, in the present embodiment, the range to be excited may be a point or a plane. Alternatively, a point to be excited may be moved (scanning). There is no specific limitation.

### 3.5 Configuration Example of a CCU 500

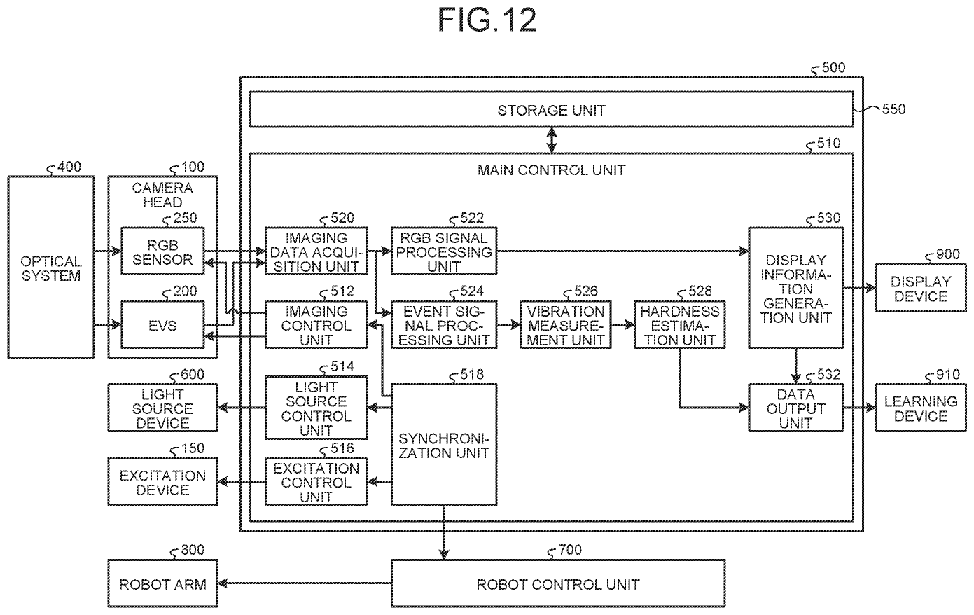

Next, a configuration example of the CCU 500 according to the present embodiment will be described with reference to FIG. 12. FIG. 12 is a block diagram illustrating an example of a functional block configuration of the CCU 500 according to the present embodiment. As illustrated in FIG. 12, the CCU 500 mainly includes a main control unit 510 and a storage unit 550. Hereinafter, functional blocks of the CCU 500 will be sequentially described.

(Main Control Unit 510)

As illustrated in FIG. 12, the main control unit 510 mainly includes an imaging control unit 512, a light source control unit 514, an excitation control unit 516, a synchronization unit 518, an imaging data acquisition unit 520, an RGB signal processing unit 522, an event signal processing unit 524, a vibration measurement unit (vibration identification unit) 526, a hardness estimation unit (estimation unit) 528, a display information generation unit (display control unit) 530, and a data output unit 532. Hereinafter, functional units of the main control unit 510 will be sequentially described.

The imaging control unit 512 can generate a control signal for controlling the EVS 200 and the RGB sensor 250 on the basis of a command output from the synchronization unit 518 (described later), and control the EVS 200 and the RGB sensor 250. At this time, in a case where an imaging condition and the like are input by the surgeon 5067, the imaging control unit 512 may generate the control signal on the basis of the input by the surgeon 5067. More specifically, in the present embodiment, on the basis of the input, a type and a state of the subject 950, a state of an image of the subject 950 which image is captured by the RGB sensor 250, brightness of irradiation light acquired from the image, a wavelength and intensity of the light emitted from the light source device 600, and the like, the imaging control unit 512 may adjust a threshold to be compared with the luminance change amount at the time of detection of an event by the EVS 200 and may enable the EVS 200 to accurately capture the displacement of the subject 950. In addition, in a case where an unintended event is detected by the EVS 200, the threshold may be set to be large, and feedback control may be performed in such a manner that only an intended event can be detected.

According to a command output from the synchronization unit 518 (described later), the light source control unit 514 can control a wavelength, a pattern, irradiation intensity, irradiation time, an irradiation interval, and the like of the light emitted from the light source device 600.

According to the command output from the synchronization unit 518 described, the excitation control unit 516 can control a frequency, an amplitude (intensity), an output pattern, a range to be excited, and the like of the vibration output from the excitation device 150. Furthermore, the excitation control unit 516 may continuously change the frequency, intensity, and the like. For example, detection by the EVS 200 may be performed while excitation intensity is gradually increased, and optimum intensity at which the subject 950 is most likely to vibrate may be identified in advance. Furthermore, after the optimum intensity is identified, detection by the EVS 200 may performed while the frequency is gradually changed, and an optimum frequency at which the subject 950 is most likely to vibrate may be identified in advance. Furthermore, for example, the characteristic of the subject 950 may be estimated by mapping (for example, with a horizontal axis as the frequency, and a vertical axis as the intensity) of a vibration state of the subject 950 observed with the excitation intensity and the excitation frequency being gradually changed.

The synchronization unit 518 can synchronize at least two of the light emission by the light source device 600, the signal detection (signal acquisition) by the EVS 200, and the excitation by the excitation device 150. For example, the synchronization unit 518 can generate a command for synchronizing operations of the imaging control unit 512, the light source control unit 514, and the excitation control unit 516 described above, output the command to the imaging control unit 512, the light source control unit 514, and the excitation control unit 516, and synchronize the imaging control unit 512, the light source control unit 514, and the excitation control unit 516. Note that in the present embodiment, synchronization is not required for all, and there is no specific limitation as long as minimum time synchronization is performed according to a situation.

The imaging data acquisition unit 520 can acquire event data and pixel signals as RAW data from the EVS 200 and the RGB sensor 250 of the camera head 100, and perform an output thereof to the RGB signal processing unit 522 and the event signal processing unit 524 (described later).

The RGB signal processing unit 522 can perform various kinds of image processing on the pixel signal that is the RAW data transmitted from the RGB sensor 250, and output a generated image to the display information generation unit 530 (described later). Examples of the image processing include various kinds of known signal processing such as development processing, image quality improving processing (such as band emphasis processing, super-resolution processing, noise reduction (NR) processing, and/or camera shake correction processing), and/or enlargement processing (electronic zoom processing).

The event signal processing unit 524 can perform various kinds of image processing on the event data and the pixel signal, which are the RAW data transmitted from the EVS 200 of the camera head 100, and output a generated image to the vibration measurement unit 526 (described later).

The vibration measurement unit 526 can extract a contour of the subject 950 and the slit pattern 960 from a plurality of the images from the event signal processing unit 524 described above by using various image recognition technologies, and identify a state of displacement (temporal change) (such as amplitude, phase, and the like) of the subject 950 or the like vibrated by the excitation device 150. Furthermore, the vibration measurement unit 526 can output the measured displacement of the subject 950 to the hardness estimation unit 528 (described later).

The hardness estimation unit 528 can estimate hardness of the subject 950 as one of the characteristics of the subject 950 on the basis of the displacement of the subject 950 from the vibration measurement unit 526 described above, and can output a result of the estimation to the display information generation unit 530 and the data output unit 532 (described later). In the present embodiment, for example, the hardness estimation unit 528 can estimate viscoelastic characteristics of the subject 950, that is, the hardness (hardness) of the subject 950 by applying the displacement (temporal change) (such as amplitude, phase, and the like) of the vibrating subject 950 or the like to a model of viscoelastic (viscosity and elasticity) impedance. Note that in the present embodiment, the hardness of the subject 950 may be estimated by an analysis of a behavior of the subject 950 due to the excitation by the excitation device 150 by utilization of a model or the like acquired by machine learning. The estimation method is not limited. Furthermore, in the present embodiment, there is no limitation to the estimation of the hardness, and the hardness estimation unit (estimation unit) 528 may estimate, for example, moisture content or the like as a parameter indicating a characteristic of an organ or the like on the basis of the displacement of the subject 950.

The display information generation unit 530 can control the display device 900 to display the image of the subject 950 (observation image) acquired by the RGB signal processing unit 522 and information based on the estimation result of the hardness estimated by the hardness estimation unit 528. For example, the display information generation unit 530 can superimpose an estimated hardness distribution (estimation result) on the image of the subject 950 and perform an output thereof to the display device 900.

The data output unit 532 can output the hardness (estimation result) estimated by the hardness estimation unit 528 to the learning device 910 and the storage unit 550. Furthermore, the data output unit 532 may output the image of the subject 950 to the learning device 910 and the storage unit 550.

(Storage Unit 550)

The storage unit 550 stores programs, information, and the like for the main control unit 510 to execute various processes. Furthermore, the storage unit 550 can also store, for example, image data or the like acquired by the RGB sensor 250 or the like of the camera head 100. Specifically, the storage unit 550 is realized by, for example, a nonvolatile memory such as a flash memory, or a storage device such as a hard disk drive (HDD).

Note that in the present embodiment, the configuration of the CCU 500 is not limited to the configuration illustrated in FIG. 12. For example, the data output unit 532 may not be provided, or a functional unit (not illustrated) may be provided.

### 3.6 Processing Method

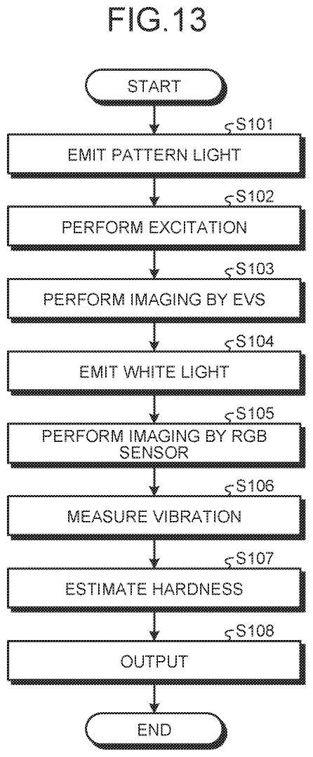

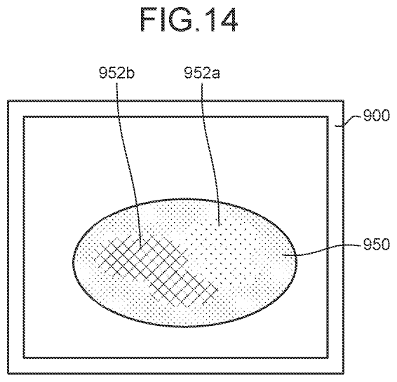

Next, an example of a processing method according to the present embodiment will be described with reference to FIG. 13 and FIG. 14. FIG. 13 is a flowchart of the processing method according to the present embodiment, and FIG. 14 is a description view for describing an example of display in the present embodiment. Specifically, as illustrated in FIG. 13, the processing method according to the present embodiment can mainly include steps from Step S101 to Step S108. Details of these steps according to the present embodiment will be described below.

First, the medical observation system 10 emits, to the subject 950, light having the slit pattern 960 by using the light source device 600 (Step S101). Then, for example, the medical observation system 10 emits an ultrasonic wave to the subject 950 while sweeping the frequency by using the excitation device 150, and excites the subject 950 (Step S102). Then, the medical observation system 10 images the subject 950 vibrated by the EVS 200 (Step S103).

Then, the medical observation system 10 stops the excitation by the excitation device 150, and emits white light to the subject 950 by using the light source device 600 (Step S104). Then, the medical observation system 10 images the subject 950 by the RGB sensor 250 (Step S105).

Then, the medical observation system 10 measures the vibration of the subject 950 by measuring the minute and high-speed displacement of the slit pattern 960 projected on the subject 950 on the basis of the image data acquired by the EVS 200 in Step S103 described above (Step S106).

Then, the medical observation system 10 estimates the hardness of the subject 950 on the basis of the behavior (displacement) of the subject 950 with respect to the vibration acquired in Step S106 described above (Step S107).

Furthermore, the medical observation system 10 superimposes the distribution of the hardness estimated in Step S107 described above on the image of the subject 950 by the RGB sensor 250 and performs a display thereof (Step S108). For example, as illustrated in FIG. 14, the medical observation system 10 displays a pattern or a color indicating a low hardness region 952a and a high hardness region 952b on the image of the subject 950 in a superimposed manner. With such display, the operator or the like can easily visually recognize the hardness of the surgical site. Furthermore, in the present embodiment, in a case where a stereoscopic image of the subject 950 is acquired, the hardness distribution may be mapped three-dimensionally. Furthermore, in the present embodiment, the image of the subject 950 by the RGB sensor 250 is not a limitation, and an image of the subject 950 by an IR sensor (not illustrated) or an SWIR sensor (not illustrated) may be displayed. There is not specifically a limitation. Furthermore, in the present embodiment, although not illustrated in FIG. 14, the medical observation system 10 may display data of the estimated hardness, an excitation method, a condition, a vibration mode, and the like. Furthermore, in the present embodiment, the acquired hardness may be fed back to the operator via a haptics device (not illustrated) worn by the operator. For example, when the surgical tool approaches the subject 950, the haptics device may vibrate and transmit the hardness of the subject 950. When the surgical tool comes into contact with the subject 950, the vibration may be stopped. Note that the detection of the approach of the surgical tool to the subject 950 can be performed according to, for example, the depth information from the depth sensor (ranging device) described above, estimation of an insertion amount of the surgical tool which amount is acquired by image recognition (such as occlusion recognition) with respect to the image from the RGB sensor 250, or the like.

Furthermore, the present embodiment may be also applied to the above-described master-slave method. Specifically, the master-slave method is, for example, a surgery supporting robot system in which surgery can be performed by a slave device (corresponding to the arm portion 5031 described above) located in an operating room when the surgeon 5067 remotely operates a master console (corresponding to the input device 5047 described above) installed at a place distant from the operating room or in the operating room. In such a case, the surgeon 5067 cannot directly touch the subject 950, and cannot know hardness of the subject 950. However, by application of the above-described present embodiment to the master-slave method, even the surgeon 5067 who exists at a remote location can recognize the hardness of the subject 950. Specifically, during surgery, when the slave device approaches the subject 950 of the surgical site, haptic feedback by the master console can be performed and the hardness of the subject 950 can be transmitted to the surgeon 5067 operating the master console. For example, resistance (impedance) of the master console to the surgeon 5067 is changed depending on the hardness of the subject 950 to which the slave device approaches. In such a manner, the surgeon 5067 can intuitively recognize the hardness of subject 950.

Furthermore, in the present embodiment, segmentation of a lesion site (region in a predetermined state) on the image of the subject 950 may be performed by utilization of the information of the hardness distribution by the identification unit (not illustrated) provided in the main control unit 510 of the CCU 500 described above. Furthermore, in the present embodiment, the identification unit may identify a position of the lesion site or estimate a state of the lesion site by analyzing the image of the subject 950, on which image the hardness distribution is superimposed, by using the model acquired by the machine learning in the learning device 910. Furthermore, in a case where the present embodiment is applied to an endoscope capable of flexibly moving in the abdominal cavity or an endoscope 5001 having an optical degree of freedom, such as a wide-angle endoscope, it is possible to determine an imaging posture of the endoscope and a range of an image cutout on the basis of the image of the subject 950 on which image the hardness distribution is superimposed. Furthermore, in the present embodiment, the image of the subject 950 on which image the hardness distribution is superimposed can be used as training data (annotation data) for machine learning in the learning device 910 by addition of information (such as a diagnostic result) by an expert.

Furthermore, in the present embodiment, in a case where the surgical tool is autonomously operated by the robot arm 800, a surgical procedure, a range of the surgical site, the operation of the robot arm 800, and the like may be determined on the basis of the information of the estimated hardness distribution. Furthermore, according to the present embodiment, it is also possible to measure a temporal change in the hardness during the surgery. Thus, in the surgery using the energy treatment tool 5021 such as an electric scalpel, it is possible to optimize contact time between the energy treatment tool 5021 and the surgical site and to make resection determination on the basis of the temporal change in the hardness. In addition, in the present embodiment, even in a case where a disturbance such as mist by the energy treatment tool 5021 is generated, since the displacement of the surgical site due to vibration can be captured by the EVS 200, the optimization of the contact time between the energy treatment tool 5021 and the surgical site and the resection determination can be robustly performed.

As described above, in the present embodiment, by applying the EVS 200, it is possible to utilize the high robustness and high time resolution in detection of a fast moving subject, which are characteristics of the EVS 200. Thus, it is possible to accurately capture minute and high-speed displacement of the subject 950 vibrated by the excitation device 150. Thus, according to the present embodiment, the hardness of the subject 950 can be measured quickly and robustly on the basis of the displacement of the subject 950 which displacement is captured by the EVS 200.

### 4. Second Embodiment

Furthermore, the embodiment of the present disclosure is not limited to the estimation of the hardness, and can also be applied to a case of determining presence or absence of contact between a surgical tool and an organ. For example, when the contact-type excitation device 150 and the subject 950 are in contact with each other, the subject 950 vibrates. Thus, conversely, when the vibration of the subject 950 is detected, the contact can be recognized. Thus, in the second embodiment, a medical observation system 10 determines presence or absence of contact on a basis of vibration of a subject 950. Note that in the following description, a case where the medical observation system 10 determines, for example, whether a distal end of a surgical tool supported by a robot arm 800 is in contact with the organ will be described as an example.

Furthermore, in the present embodiment described below, it is assumed that the above-described contact-type excitation device 150 is provided at a distal end portion of the surgical tool supported by the robot arm 800. In addition, since a configuration of the medical observation system 10 is similar to that of the first embodiment, description thereof is omitted here.

<4.1 Configuration Example of a CCU 500a>

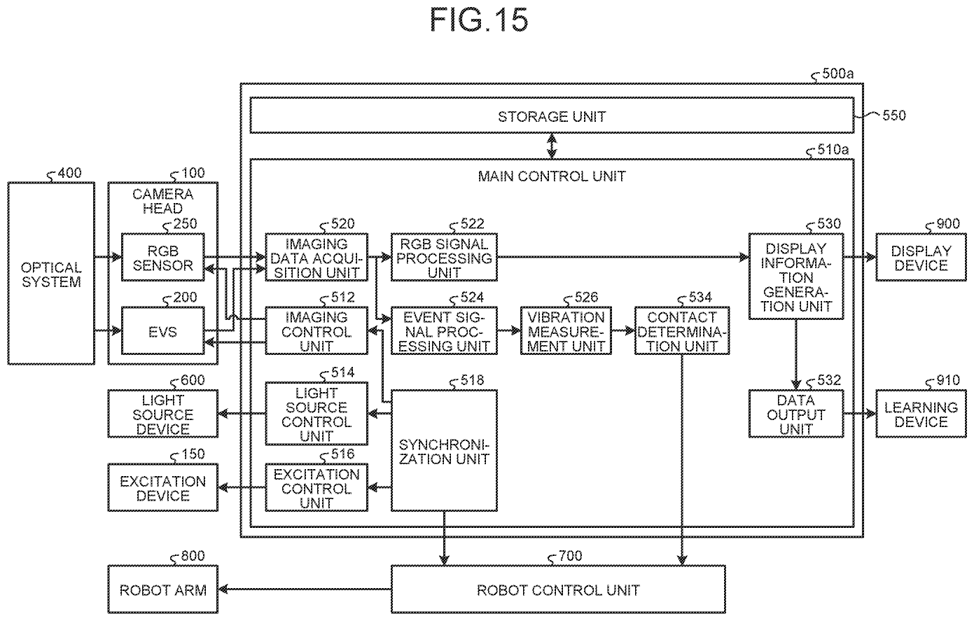

First, a configuration example of the CCU 500a according to the present embodiment will be described with reference to FIG. 15. FIG. 15 is a block diagram illustrating an example of a functional block configuration of the CCU 500a according to the present embodiment. As illustrated in FIG. 15, similarly to the first embodiment, a CCU 500 mainly includes a main control unit 510a and a storage unit 550. In the following description, since the storage unit 550 is similar to that of the first embodiment, only the main control unit 510a will be described.

(Main Control Unit 510a)

As illustrated in FIG. 15, similarly to the first embodiment, the main control unit 510a mainly includes an imaging control unit 512, a light source control unit 514, an excitation control unit 516, a synchronization unit 518, an imaging data acquisition unit 520, an RGB signal processing unit 522, an event signal processing unit 524, a vibration measurement unit 526, a display information generation unit (output unit) 530, and a data output unit 532. Furthermore, in the present embodiment, the main control unit 510a includes a contact determination unit (estimation unit) 534. In the following description, description of each functional unit common to the first embodiment will be omitted, and only the contact determination unit (estimation unit) 534 will be described.

The contact determination unit 534 can determine presence or absence of contact between a subject 950 and a surgical tool, to which the excitation device 150 is attached, on the basis of displacement of the subject 950 from the vibration measurement unit 526 described above. Furthermore, in the present embodiment, the contact determination unit 534 may estimate a range of contact with the surgical tool and a degree of the contact on the basis of a range of the subject 950 with displacement. Furthermore, the contact determination unit 534 can output a result of the determination to a robot control unit 700.

Note that in the present embodiment, the configuration of the CCU 500a is not limited to the configuration illustrated in FIG. 15. For example, the data output unit 532 may not be provided, or a functional unit (not illustrated) may be provided.

Furthermore, in the present embodiment, the excitation device 150 is not limited to being provided at the distal end portion of the surgical tool supported by the robot arm 800, and may be provided, for example, in a camera head 100 or an optical system 400 supported by the robot arm 800. In this case, the medical observation system 10 determines whether the camera head 100 or the optical system 400 is in contact with the subject 950. For example, in a case where the excitation device 150 is provided in the camera head 100 or the optical system 400, an image of a surgical site becomes unclear due to mist generated from the surgical site, and presence or absence of contact with the surgical site cannot be determined from the image in some cases. However, according to the present embodiment, even in a case where the mist is generated, displacement of the surgical site due to vibration can be captured by an EVS 200, whereby the presence or absence of the contact with the surgical site can be determined.

<4.2 Processing Method>

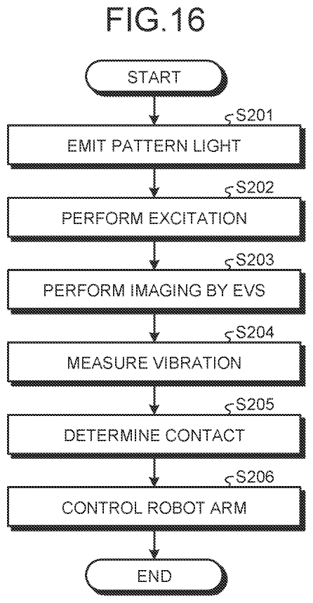

Next, an example of a processing method according to the present embodiment will be described with reference to FIG. 16. FIG. 16 is a flowchart of the processing method according to the present embodiment. Specifically, as illustrated in FIG. 16, the processing method according to the present embodiment can mainly include steps from Step S201 to Step S206. Details of these steps according to the present embodiment will be described below.

First, the medical observation system 10 emits, to the subject 950, light having a slit pattern 960 by using a light source device 600 similarly to Step S101 of the first embodiment (Step S201). Then, the medical observation system 10 attempts to excite the subject 950 by using the excitation device 150 at the distal end of the surgical tool (Step S202). Note that in the present embodiment, it is assumed that the excitation device 150 attempts to constantly perform excitation during surgery. Then, the medical observation system 10 images the subject 950 by the EVS 200 (Step S203).

Then, the medical observation system 10 measures presence or absence of the vibration of the subject 950 by measuring displacement of the slit pattern 960, which is projected on the subject 950, on the basis of image data acquired by the EVS 200 in Step S103 described above (Step S204).

Then, the medical observation system 10 determines the presence or absence of contact with the subject 950 on the basis of a behavior (displacement) of the subject 950 with respect to the vibration acquired in Step S204 described above (Step S205).

Furthermore, on the basis of a result of the determination in Step S205 described above, the medical observation system 10 controls the robot arm 800 that supports the surgical tool (Step S206). For example, in a case where unnecessary contact between the subject 950 and the surgical tool is detected, the medical observation system 10 retracts the robot arm 800 supporting the surgical tool from the subject 950 or stops operation of the surgical tool itself. Furthermore, for example, in a case where non-contact between the subject 950 and the camera head 100 or the optical system 400 is detected, the medical observation system 10 controls the robot arm 800 and retracts the camera head 100 or the optical system 400 from the subject 950. In the present embodiment, since the contact can be quickly and reliably detected and the robot arm 800 can be immediately controlled, safety of the operation of the robot arm 800 can be improved.

As described above, in the present embodiment, by applying the EVS 200, it is possible to utilize high robustness and high time resolution in detection of a fast moving subject, which are characteristics of the EVS 200. Thus, it is possible to accurately capture minute and high-speed displacement of the subject 950 vibrated by the excitation device 150. Thus, according to the present embodiment, it is possible to quickly and robustly determine the presence or absence of the contact with the subject 950 on the basis of the displacement of the subject 950 captured by the EVS 200.

### 5. Conclusion

As described above, according to embodiments of the present disclosure, by applying the EVS 200, it is possible to utilize the high robustness and high time resolution in detection of a fast moving subject, which are characteristics of the EVS 200. Thus, it is possible to accurately capture minute and high-speed displacement of the subject 950 vibrated by the excitation device 150. Thus, according to the present embodiment, the hardness of the subject 950 and the presence or absence (state) of the contact with the subject 950 can be quickly and robustly measured on the basis of the displacement of the subject 950 captured by the EVS 200.

Note that in the embodiment of the present disclosure described above, an imaging target is not limited to be in an abdominal cavity, and may be a biological tissue, a fine mechanical structure, or the like, and is not specifically limited. Furthermore, the above-described embodiments of the present disclosure are not limited to application to a use of medical care, research, or the like, and can be applied to an observation device that performs highly accurate analysis or the like by using an image. Thus, the medical observation system 10 described above can be used as an observation system (observation device).

Furthermore, the medical observation system 10 described above can be used as a rigid endoscope, a flexible endoscope, an exoscope, a microscope, or the like, and may not include the robot arm 800 or may include only the EVS 200, and a configuration thereof is not specifically limited.

### 6. Hardware Configuration

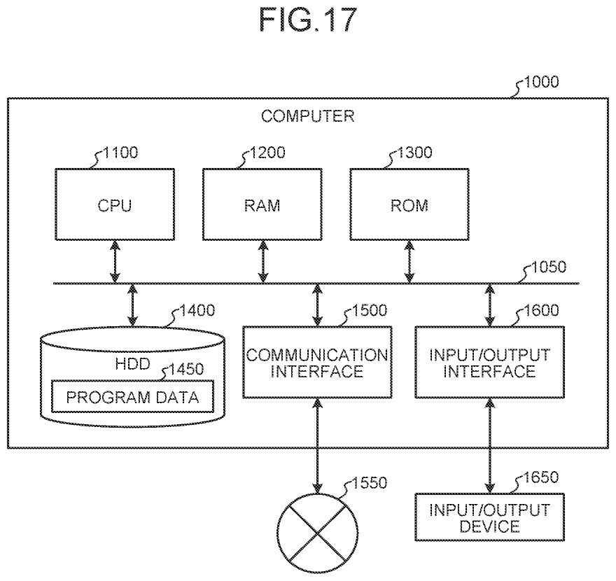

The information processing device such as the CCU 500 according to each of the embodiments described above is realized by, for example, a computer 1000 having a configuration in a manner illustrated in FIG. 17. Hereinafter, a CCU 500 according to an embodiment of the present disclosure will be described as an example. FIG. 17 is a hardware configuration diagram illustrating an example of a computer that realizes the CCU 500 according to the embodiment of the present disclosure. The computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates on the basis of programs stored in the ROM 1300 or the HDD 1400, and controls each unit. For example, the CPU 1100 expands the programs, which are stored in the ROM 1300 or the HDD 1400, in the RAM 1200 and executes processing corresponding to the various programs.

The ROM 1300 stores a boot program such as a basic input output system (BIOS) executed by the CPU 1100 during activation of the computer 1000, a program that depends on hardware of the computer 1000, and the like.

The HDD 1400 is a computer-readable recording medium that non-temporarily records a program executed by the CPU 1100, data used by the program, and the like. Specifically, the HDD 1400 is a recording medium that records a program for the medical observation system 10 according to the present disclosure which program is an example of program data 1450.

The communication interface 1500 is an interface with which the computer 1000 is connected to an external network 1550 (such as the Internet). For example, the CPU 1100 receives data from another equipment or transmits data generated by the CPU 1100 to another equipment via the communication interface 1500.

The input/output interface 1600 is an interface to connect an input/output device 1650 and the computer 1000. For example, the CPU 1100 receives data from an input device such as a keyboard or a mouse via the input/output interface 1600. Also, the CPU 1100 transmits data to an output device such as a display, a speaker, or a printer via the input/output interface 1600. Furthermore, the input/output interface 1600 may function as a medium interface that reads a program or the like recorded on a computer-readable predetermined recording medium (medium). The medium is, for example, an optical recording medium such as a digital versatile disc (DVD) or phase change rewritable disk (PD), a magneto-optical recording medium such as a magneto-optical disk (MO), a tape medium, a magnetic recording medium, a semiconductor memory, or the like.

For example, in a case where the computer 1000 functions as the CCU 500 according to the embodiment of the present disclosure, the CPU 1100 of the computer 1000 realizes a function of controlling the medical observation system 10 by executing a program loaded on the RAM 1200. In addition, the HDD 1400 may store a program for controlling the medical observation system 10 according to the embodiment of the present disclosure. Note that the CPU 1100 reads the program data 1450 from the HDD 1400 and performs execution thereof, but may acquire information processing programs from another device via the external network 1550 in another example.

Furthermore, the CCU 500 according to the present embodiment may be applied to a system including a plurality of devices on the premise of connection to a network (or communication between devices), such as cloud computing.

An example of the hardware configuration of the CCU 500 has been described above. Each of the above-described components may be configured by utilization of a general-purpose member, or may be configured by hardware specialized for a function of each component. Such a configuration can be appropriately changed according to a technical level at the time of implementation.

### 7. Supplementary Note

Note that the embodiment of the present disclosure described above can include, for example, an information processing method executed by the medical observation system 10 as described above, a program for causing the medical observation system 10 to function, and a non-transitory tangible medium in which the program is recorded. Furthermore, the program may be distributed via a communication line (including wireless communication) such as the Internet.

Furthermore, each step in the processing method of the embodiment of the present disclosure described above may not necessarily be processed in the described order. For example, the steps may be processed in appropriately changed order. In addition, the steps may be processed partially in parallel or individually instead of being processed in time series. Furthermore, the processing of each step is not necessarily performed according to the described method, and may be performed by another method by another functional unit, for example.

Also, among the pieces of processing described in each of the above embodiments, all or a part of the processing described to be automatically performed can be manually performed, or all or a part of the processing described to be manually performed can be automatically performed by a known method. In addition, the processing procedures, specific names, and information including various kinds of data or parameters described in the above document or in the drawings can be arbitrarily changed unless otherwise specified. For example, various kinds of information illustrated in each of the drawings are not limited to the illustrated information.

Also, each component of each of the illustrated devices is a functional concept, and does not need to be physically configured in the illustrated manner. That is, a specific form of distribution/integration of each device is not limited to what is illustrated in the drawings, and a whole or part thereof can be functionally or physically distributed/integrated in an arbitrary unit according to various loads and usage conditions.

Preferred embodiments of the present disclosure have been described in detail in the above with reference to the accompanying drawings. However, a technical scope of the present disclosure is not limited to such examples. It is obvious that a person having ordinary knowledge in the technical field of the present disclosure can conceive various alterations or modifications within the scope of the technical idea described in the claims, and it should be understood that these alterations or modifications naturally belong to the technical scope of the present disclosure.

In addition, the effects described in the present specification are merely illustrative or exemplary, and are not restrictive. That is, in addition to the above effects or instead of the above effects, the technology according to the present disclosure can exhibit a different effect obvious to those skilled in the art from the description of the present specification.

Note that the present technology can also have the following configurations.
(1) An in-vivo observation system comprising:
   an excitation device that vibrates an object in a living body;
   an event vision sensor that detects, as an event, a change due to the vibration in a luminance value of light emitted from the object; and
   an estimation unit that estimates a characteristic of the object on a basis of sensing data from the event vision sensor.
(2) The in-vivo observation system according to (1), wherein the event vision sensor includes
   a pixel array unit including a plurality of pixels arrayed in a matrix, and
   an event detection unit that detects, in each of the pixels, that a luminance change amount due to light emitted from the object exceeds a predetermined threshold.
(3) The in-vivo observation system according to (2), further comprising
   an image sensor that generates an observation image from the light emitted from the object.
(4) The in-vivo observation system according to (3), wherein the event vision sensor and the image sensor are provided on different substrates.
(5) The in-vivo observation system according to (3), wherein the event vision sensor and the image sensor are provided on a same substrate.
(6) The in-vivo observation system according to any one of (3) to (5), further comprising a display control unit that controls a display device to display the observation image of the object by the image sensor and information based on an estimation result of the estimation unit.
(7) The in-vivo observation system according to any one of (3) to (6), further comprising a light source that emits light to an inside of the living body.
(8) The in-vivo observation system according to (7), wherein the event vision sensor and the light source are coaxially provided with respect to the object.
(9) The in-vivo observation system according to (7) or (8), further comprising a synchronization unit that synchronizes at least two of light emission by the light source, signal acquisition by the event vision sensor, and excitation by the excitation device.
(10) The in-vivo observation system according to any one of (7) to (9), wherein the light source emits first light for identifying the characteristic of the object and second light for generating the observation image of the object.
(11) The in-vivo observation system according to (10), wherein the light source alternately emits the first light and the second light to the object.
(12) The in-vivo observation system according to (10) or (11), wherein the first light and the second light have wavelength bands different from each other.
(13) The in-vivo observation system according to any one of (10) to (12), wherein the light source emits at least one of visible light, infrared light, and short-wavelength infrared light to the object.
(14) The in-vivo observation system according to any one of (10) to (13), wherein the light source projects, onto the object, light having a predetermined pattern as the first light.
(15) The in-vivo observation system according to (14), wherein the predetermined pattern is any one of a slit pattern, a lattice pattern, or a moire fringe.
(16) The in-vivo observation system according to any one of (1) to (15), wherein the excitation device includes a vibrator that comes into contact with the object and applies vibration.
(17) The in-vivo observation system according to any one of (1) to (15), wherein the excitation device emits an ultrasonic wave or light to the object.
(18) The in-vivo observation system according to any one of (1) to (17), wherein the excitation device is provided at a distal end of the event vision sensor or a surgical tool.
(19) The in-vivo observation system according to any one of (1) to (18), further comprising
   an excitation device control unit that controls the excitation device, wherein
   the excitation device control unit changes at least one of a frequency of an output from the excitation device, an output pattern from the excitation device, or a range of the object to be excited.
(20) The in-vivo observation system according to any one of (1) to (19), wherein the estimation unit estimates hardness or moisture content of the object.
(21) The in-vivo observation system according to (20), further comprising an identification unit that identifies a region in a predetermined state in the object on a basis of a result of the estimation.
(22) An in-vivo observation system comprising:
   an excitation device that vibrates an object in a living body;
   an event vision sensor that detects, as an event, a change due to the vibration in a luminance value of light emitted from the object; and
   an estimation unit that estimates presence or absence of contact between the object and the excitation device on a basis of sensing data from the event vision sensor.
(23) The in-vivo observation system according to any one of (20) to (22), further comprising a vibration identification unit that identifies the vibration of the object due to the excitation device on a basis of the sensing data from the event vision sensor.
(24) The in-vivo observation system according to any one of (1) to (23), wherein the event vision sensor images an inside of an abdominal cavity of the living body.
(25) The in-vivo observation system according to any one of (1) to (24), wherein the system is any one of an endoscope, an exoscope, or a microscope.
(26) The in-vivo observation system according to (1), further comprising a robot arm that supports the event vision sensor or a surgical tool.
(27) An observation system comprising:
   an excitation device that vibrates an object;
   an event vision sensor that detects, as an event, a change due to the vibration in a luminance value of light emitted from the object; and
   an estimation unit that estimates a characteristic of the object on a basis of sensing data from the event vision sensor.
(28) An in-vivo observation method comprising:
   vibrating an object in a living body by using an excitation device;
   detecting a change due to the vibration in a luminance value of light, which is emitted from the object, as an event by using an event vision sensor; and
   estimating, by a computer, a characteristic of the object on a basis of sensing data from the event vision sensor.
(29) An in-vivo observation device comprising:
   an excitation device that vibrates an object in a living body;
   an event vision sensor that detects, as an event, a change due to the vibration in a luminance value of light emitted from the object; and
   an estimation unit that estimates a characteristic of the object on a basis of sensing data from the event vision sensor.

### REFERENCE SIGNS LIST

10 MEDICAL OBSERVATION SYSTEM
100, 100a CAMERA HEAD
150, 150a, 150b, 150c EXCITATION DEVICE
200 EVS
211 DRIVE CIRCUIT
212 SIGNAL PROCESSING UNIT
213 ARBITER UNIT
214 COLUMN PROCESSING UNIT
250 RGB SENSOR
260 PRISM
300 PIXEL ARRAY UNIT
302 PIXEL
304 LIGHT RECEIVING UNIT
306 PIXEL SIGNAL GENERATION UNIT
308 DETECTION UNIT
400 OPTICAL SYSTEM
500, 500a CCU
510, 510a MAIN CONTROL UNIT
512 IMAGING CONTROL UNIT
514 LIGHT SOURCE CONTROL UNIT
516 EXCITATION CONTROL UNIT
518 SYNCHRONIZATION UNIT
520 IMAGING DATA ACQUISITION UNIT
522 RGB SIGNAL PROCESSING UNIT
524 EVENT SIGNAL PROCESSING UNIT
526 VIBRATION MEASUREMENT UNIT
528 HARDNESS ESTIMATION UNIT
530 DISPLAY INFORMATION GENERATION UNIT
532 DATA OUTPUT UNIT
534 CONTACT DETERMINATION UNIT
550 STORAGE UNIT
600 LIGHT SOURCE DEVICE
700 ROBOT CONTROL UNIT
800 ROBOT ARM
900 DISPLAY DEVICE
910 LEARNING DEVICE
950 SUBJECT
952a, 952b REGION
960, 960a, 960b SLIT PATTERN

1. An in-vivo observation system comprising:
an excitation device that vibrates an object in a living body;
an event vision sensor that detects, as an event, a change due to the vibration in a luminance value of light emitted from the object; and
an estimation unit that estimates a characteristic of the object on a basis of sensing data from the event vision sensor.

2. The in-vivo observation system according to claim 1, wherein
the event vision sensor includes
a pixel array unit including a plurality of pixels arrayed in a matrix, and
an event detection unit that detects, in each of the pixels, that a luminance change amount due to light emitted from the object exceeds a predetermined threshold.

3. The in-vivo observation system according to claim 2, further comprising
an image sensor that generates an observation image from the light emitted from the object.

4. The in-vivo observation system according to claim 3, wherein the event vision sensor and the image sensor are provided on different substrates.

5. The in-vivo observation system according to claim 3, wherein the event vision sensor and the image sensor are provided on a same substrate.

6. The in-vivo observation system according to claim 3, further comprising a display control unit that controls a display device to display the observation image of the object by the image sensor and information based on an estimation result of the estimation unit.

7. The in-vivo observation system according to claim 3, further comprising a light source that emits light to an inside of the living body.

8. The in-vivo observation system according to claim 7, wherein the event vision sensor and the light source are coaxially provided with respect to the object.

9. The in-vivo observation system according to claim 7, further comprising a synchronization unit that synchronizes at least two of light emission by the light source, signal acquisition by the event vision sensor, and excitation by the excitation device.

10. The in-vivo observation system according to claim 7, wherein the light source emits first light for identifying the characteristic of the object and second light for generating the observation image of the object.

11. The in-vivo observation system according to claim 10, wherein the light source alternately emits the first light and the second light to the object.

12. The in-vivo observation system according to claim 10, wherein the first light and the second light have wavelength bands different from each other.

13. The in-vivo observation system according to claim 10, wherein the light source emits at least one of visible light, infrared light, and short-wavelength infrared light to the object.

14. The in-vivo observation system according to claim 10, wherein the light source projects, onto the object, light having a predetermined pattern as the first light.

15. The in-vivo observation system according to claim 14, wherein the predetermined pattern is any one of a slit pattern, a lattice pattern, or a moire fringe.

16. The in-vivo observation system according to claim 1, wherein the excitation device includes a vibrator that comes into contact with the object and applies vibration.

17. The in-vivo observation system according to claim 1, wherein the excitation device emits an ultrasonic wave or light to the object.

18. The in-vivo observation system according to claim 1, wherein the excitation device is provided at a distal end of the event vision sensor or a surgical tool.

19. The in-vivo observation system according to claim 1, further comprising
an excitation device control unit that controls the excitation device, wherein
the excitation device control unit changes at least one of a frequency of an output from the excitation device, an output pattern from the excitation device, or a range of the object to be excited.

20. The in-vivo observation system according to claim 1, wherein the estimation unit estimates hardness or moisture content of the object.

21. The in-vivo observation system according to claim 20, further comprising an identification unit that identifies a region in a predetermined state in the object on a basis of a result of the estimation.

22. An in-vivo observation system comprising:
an excitation device that vibrates an object in a living body;
an event vision sensor that detects, as an event, a change due to the vibration in a luminance value of light emitted from the object; and
an estimation unit that estimates presence or absence of contact between the object and the excitation device on a basis of sensing data from the event vision sensor.

23. The in-vivo observation system according to claim 20, further comprising a vibration identification unit that identifies the vibration of the object due to the excitation device on a basis of the sensing data from the event vision sensor.

24. The in-vivo observation system according to claim 1, wherein the event vision sensor images an inside of an abdominal cavity of the living body.

25. The in-vivo observation system according to claim 1, wherein the system is any one of an endoscope, an exoscope, or a microscope.

26. The in-vivo observation system according to claim 1, further comprising a robot arm that supports the event vision sensor or a surgical tool.

27. An observation system comprising:
an excitation device that vibrates an object;
an event vision sensor that detects, as an event, a change due to the vibration in a luminance value of light emitted from the object; and
an estimation unit that estimates a characteristic of the object on a basis of sensing data from the event vision sensor.

28. An in-vivo observation method comprising:
vibrating an object in a living body by using an excitation device;
detecting a change due to the vibration in a luminance value of light, which is emitted from the object, as an event by using an event vision sensor; and
estimating, by a computer, a characteristic of the object on a basis of sensing data from the event vision sensor.

29. An in-vivo observation device comprising:
an excitation device that vibrates an object in a living body;
an event vision sensor that detects, as an event, a change due to the vibration in a luminance value of light emitted from the object; and
an estimation unit that estimates a characteristic of the object on a basis of sensing data from the event vision sensor.
